(19) United States
(12) Patent Application Publication
(10) Pub. No.: US 2006/0233683 A1
(43) Pub. Date: Oct. 19, 2006

(54) Means for killing pathogens in atmosphere and on artificial and natural surfaces including skin

(76) Inventor: David Murray MacDonald, Irthington (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 11/376,954

(22) Filed: Mar. 16, 2006

Related U.S. Application Data
(63) Continuation of application No. PCT/GB04/03949, filed on Sep. 16, 2004.

(30) Foreign Application Priority Data
Sep. 16, 2003 (GB) 0321665.2

(51) Int. Cl. B01J 19/08

(52) U.S. Cl. 422/186.07

(57) ABSTRACT

An apparatus for generating hydroxyl radicals comprises sources of oxygen and olefin. Oxygen is delivered to an ozone generator. Olefin and generated ozone are mixed producing hydroxyl radicals.

### CROSS-REFERENCE TO RELATED APPLICATIONS

This application is a continuation of International Application No. PCT/GB2004/003949, filed Sep. 16, 2004, which claims priority from U.K. Patent Application No. 0321665.2, filed Sep. 16, 2003. The disclosures of both applications are incorporated herein by reference.

### BACKGROUND OF THE INVENTION

The use of bactericides to disinfect rooms and surfaces gives rise to increasingly prevalent populations of bacteria that are resistant to antibiotics and as a result difficult to treat. Anti-biotics are ineffective against viruses. The problems that result are cross infection especially in hospitals, nursing homes, surgeries, aircraft or trains, food preparation units and all spaces here fresh air is limited in its access and where numbers of people are normally present. Where the areas are open to fresh air, the natural systems that exist for the control of pathogen populations (namely, the production of hydroxyl radicals from the decay of atmospheric ozone in the presence of olefins) can function. In any area where fresh air is limited, the population of pathogens can rise. This is especially acute in hospitals where hospital acquired infections are endemic, affecting up to 10% or more of all patients. Such infections can be acquired in a number of ways and the reduction in concentration of killed pathogens compared with fresh or open air has the added effect of reduced immunity. Such immunity is caused by pulmonary inoculation as dead pathogens are absorbed through the alveolar system of the lungs.

The present invention utilizes the natural systems used to control pathogens both in atmosphere and in mammals to avoid the use of bactericides. That system relies on the production of short-lived hydroxyl radicals (OH) that react with the phospho-lipid plasma of the pathogen to induce peroxidation in the pathogen bringing about its death.

The natural system referred to was first discovered in the 1960's by researchers at Porton Down in the United Kingdom and TNO in the Netherlands who were investigating how pathogens died in air. They found that the primary method of control is the release of hydroxyl radicals. They found that pathogens died in air at rates that varied depending on weather, airborne pollutants and wind direction. They demonstrated that there was a factor in the atmosphere that destroyed pathogens and called that the Open Air Factor. It was later established that the Open Air Factor was formed by the action of constituents of the atmosphere with a range of olefins, both synthetic and naturally occurring. Terpinenes were of particular efficacy, terpinenes being associated with the scent of flowers or of pine trees. Dutch research showed that a threshold level of ozone concentration of 80 ppb was required, with the presence of olefins, for the Open Air Factor to become fully effective.

In the research at Porton Down referred to above it was found that the Open Air Factor was markedly reduced in a closed chamber. At the time it was thought that the Open Air Factor was absorbed on the surface of the container. However, it is more likely that the effects of the metal container surfaces were to react with free radicals in preference to the free radicals reacting with cell surfaces, resulting in a reduction in efficacy of the Open Air Factor.

### SUMMARY OF THE INVENTION

In the present invention the hydroxyl radical can conveniently be produced by causing ozone to decay to normal oxygen through the reaction of ozone with an olefin. In the natural state these olefins are naturally occurring substances such as terpinenes produced by the metabolism of plants and flowers, although synthetic olefins can be used.

Suitable olefins include the naturally occurring olefins alpha-terpinene, delta-limonene, myrcen, and the synthetic olefins pentene, cyclohexene and butene.

The present invention provides a means of introducing raised concentrations of hydroxyl radicals to bring about the disinfection of surfaces and atmospheres. The life of a hydroxyl radical is extremely short, in normal events less than seconds, and as such the hydroxyl radical must be produced close to the target pathogen. It is therefore necessary that the apparatus for producing the hydroxyl radicals is portable. The present invention consists of a supply of oxygen, which is passed through a means of converting oxygen, or part of the flow of oxygen, to ozone. The ozone is then mixed with a source of olefin, typically but not solely an olefin such as trans 2-butene, in proximity to the pathogen. In order to accelerate the process, it is desirable that the oxygen stream, or the mixture of ozone and oxygen, be rendered humid and that this humidity encompasses a ferrous salt.

According to one aspect of the invention, there is provided an apparatus as specified in Claim 1.

According to another aspect of the invention, there is provided an ozone generator as specified in Claim 18.

According to another aspect of the invention, there is provided a method of killing pathogens as specified in Claim 26.

Various objects and advantages of this invention will become apparent to those skilled in the art from the following detailed description of the preferred embodiments, when read in light of the accompanying drawings.

### BRIEF DESCRIPTION OF THE DRAWINGS

FIG. 1 is a schematic representation of an embodiment of apparatus according to the invention;

FIG. 2a is an exploded view illustrating parts of the apparatus illustrated in FIG. 1;

FIG. 2b is a cross-sectional elevation of the apparatus illustrated in FIG. 1;

FIG. 2c is a plan view of a part of the apparatus illustrated in FIG. 2b;

FIG. 2d is a side view of an ozone generator;

FIG. 2e is a side view of an element of the ozone generator illustrated in FIG. 2d;

FIG. 2f is a schematic representation of the ozone generator illustrated in FIG. 2d;

FIG. 2g is a schematic representation of an alternative ozone generator;

FIG. 2h is an end view of the ozone generator illustrated in FIG. 2g; and

FIG. 2i is a cross-sectional elevation of a part of the ozone generator illustrated in FIGS. 2g and 2h;

FIG. 3a is a plan view of a hood;

FIG. 3b is a front view of the hood illustrated in FIG. 3a;

FIG. 4a is a cross-sectional elevation of a plate for mounting a hand shroud;

FIG. 4b is a plan view of the plate shown in FIG. 4a;

FIG. 4c is a plan view of a hand shroud;

FIG. 4d is a front elevation of the hand shroud illustrated in FIG. 4c;

FIG. 4e is a side elevation of the hand shroud illustrated in FIGS. 4c and 4d;

FIG. 4f is a plan view of a clamp;

FIG. 4g is a front elevation of the clamp illustrated in FIG. 4f; and

FIG. 5 is a cross-sectional elevation of another embodiment of a device for generating hydroxyl radicals according to the invention.

### DETAILED DESCRIPTION OF THE PREFERRED EMBODIMENTS

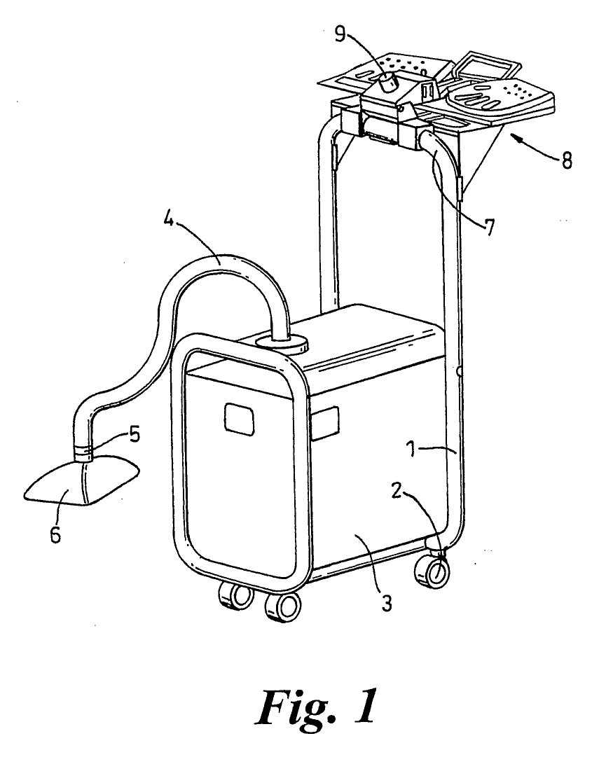

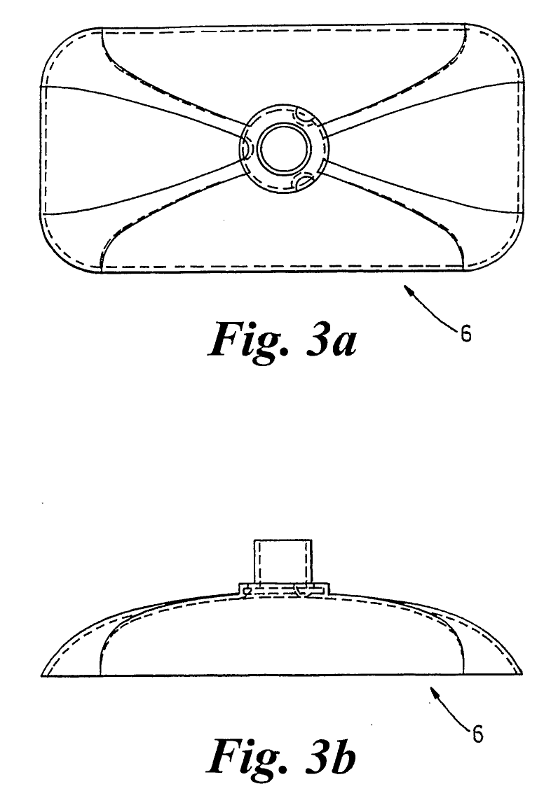

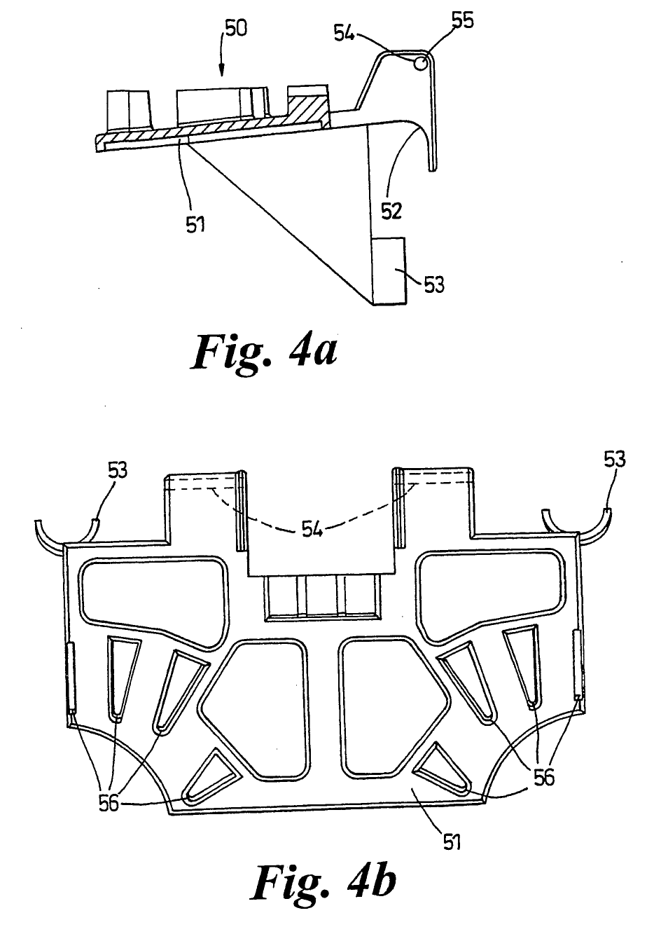

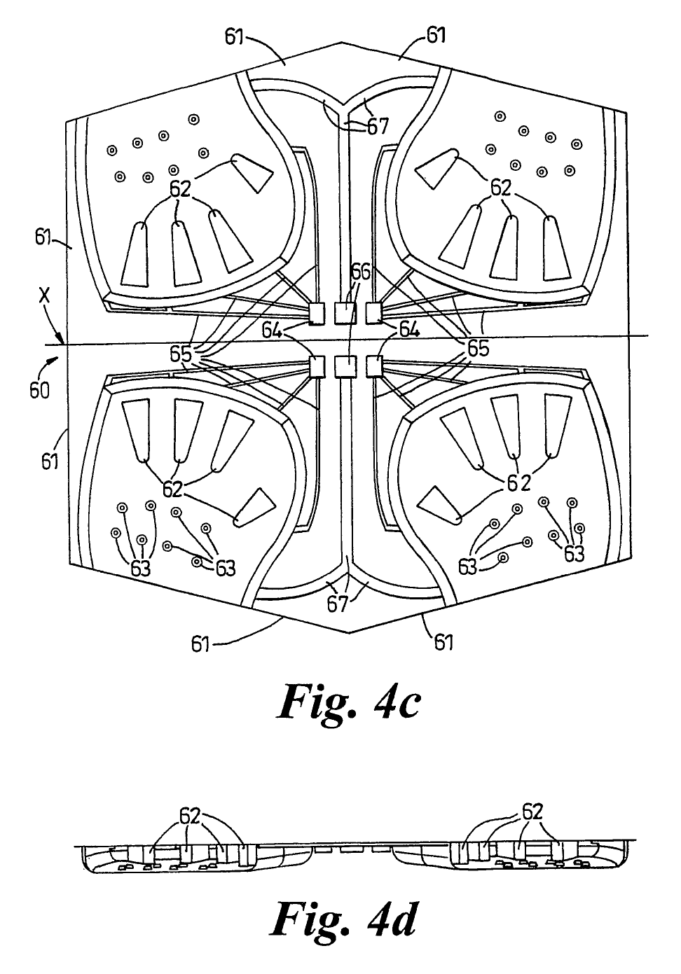

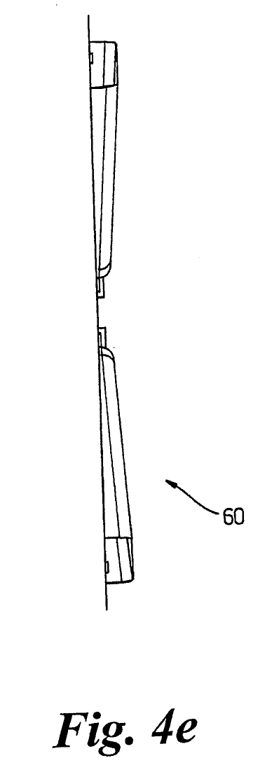

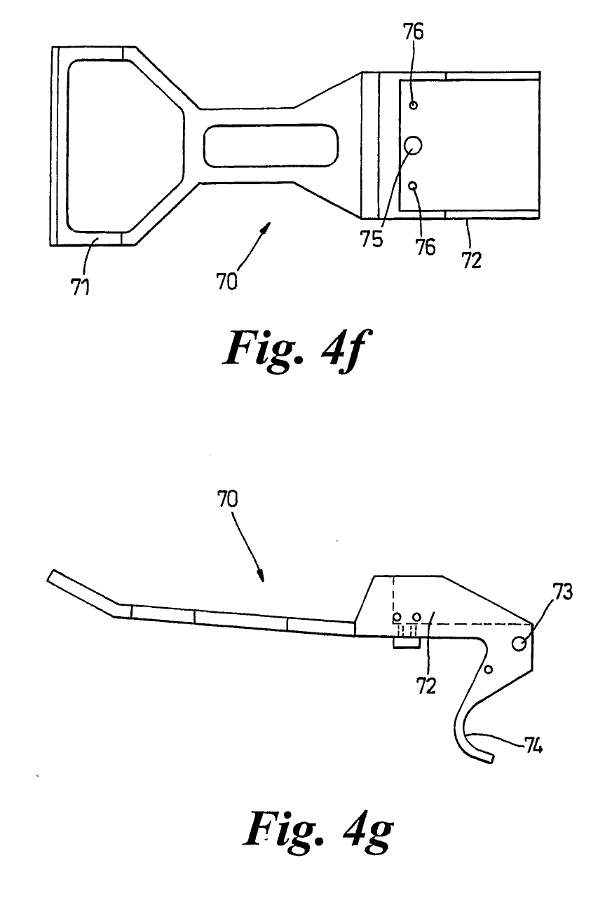

Referring now to FIG. 1, there is shown an apparatus for generating hydroxyl radicals for use in hospital environments. The apparatus comprises a chassis 1 mounted on wheels 2. The chassis 1 mounts a box like cage 3 which houses supplies of oxygen and olefin, and ozone generator. The cage 3 includes an outlet through which ozone and olefin are directed. A tube 4 is attached to the outlet. The tube 4 has at its free end a fitting 5 for attachment of a hood 6 thereto. Ozone and olefin are mixed in the hood. The hood and its use will be described in greater detail in relation to FIGS. 3a and 3b. The hood 6 can be removed from the fitting 5. This allows the fitting 5 to be attached to an inlet 9 of a hand shroud 8 mounted on a handle member 7 of the chassis 1. The shroud and its function will be described in greater detail with reference to FIGS. 4a to 4f.

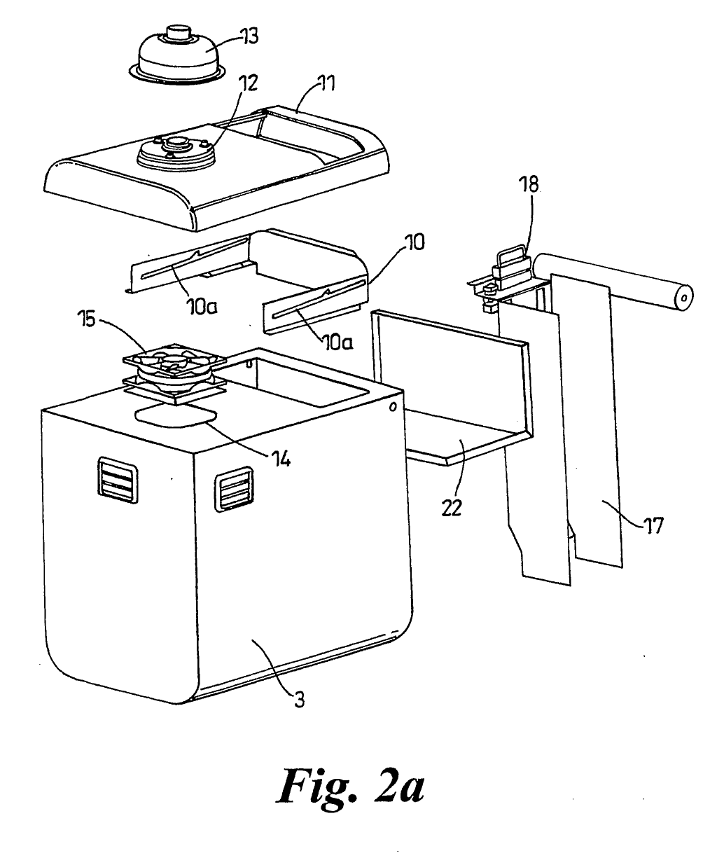

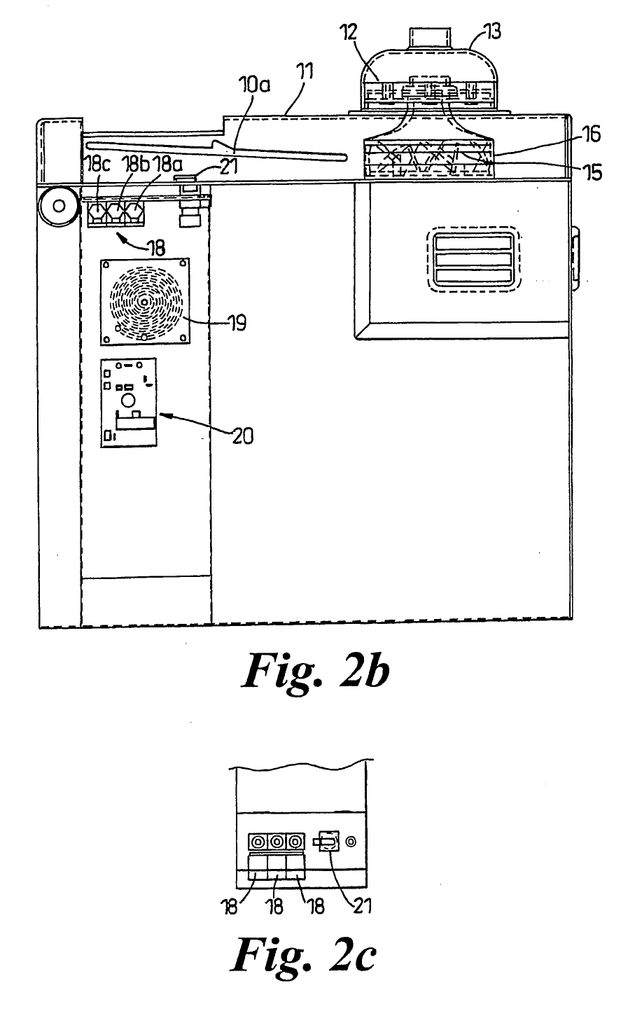

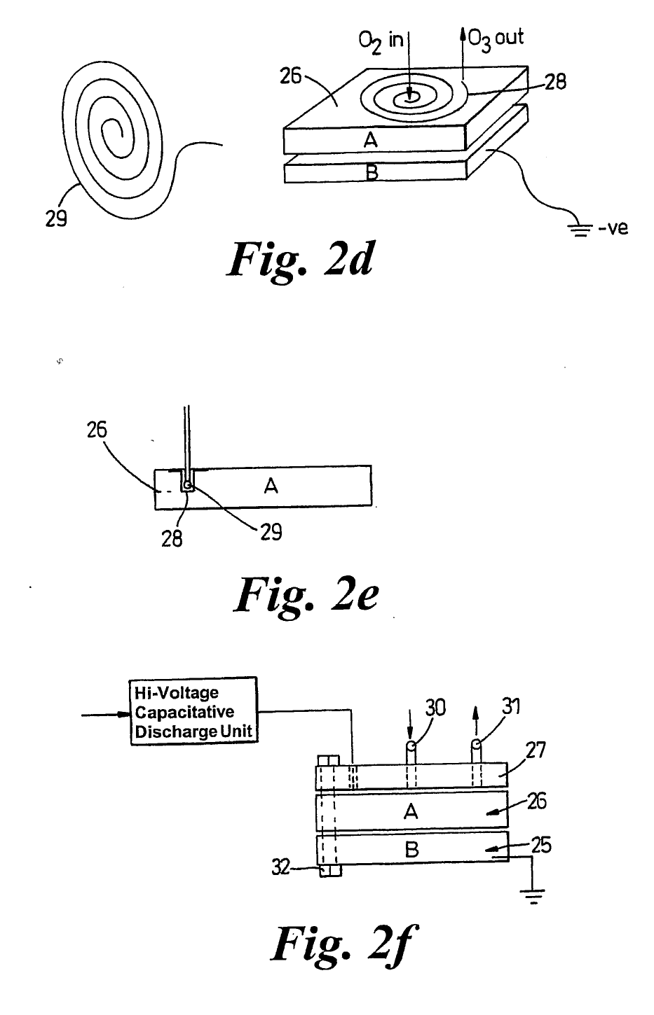

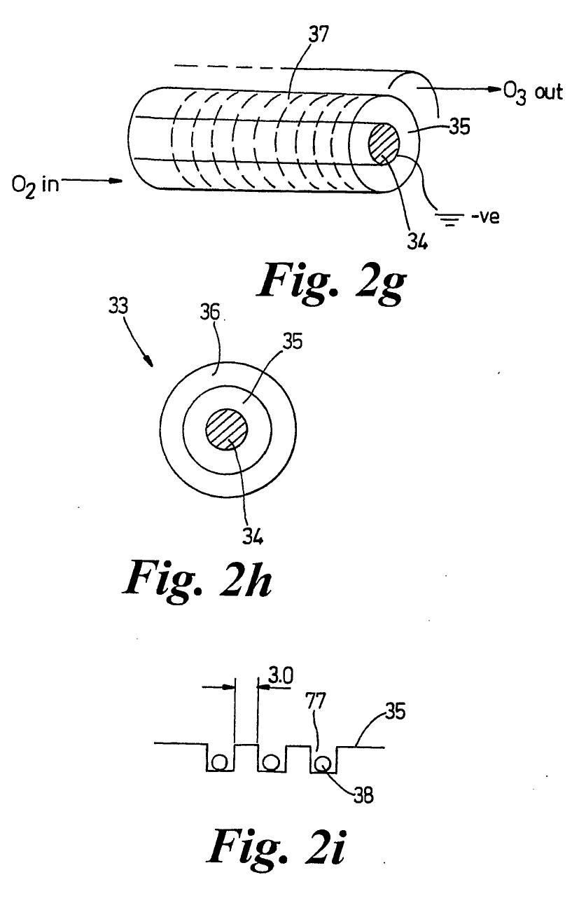

Referring now to FIGS. 2a and 2b, the cage 3 includes an opening 14 in which a fan 15 may be is mounted. A U-shaped member 10 is attached to the cage 3 using plastic rivets. Two sides of the U-shaped member include a slide rail 10a. A lid 11 fits onto the cage 3 by means of the slide rails 10a. The lid 11, which may be vacuum formed from plastics material, mounts a valve disc 12 which is covered by a hood 13. A separate lid slides on the slide rails: mounted in the lid is an electrical switch operated by a key and so arranged that the lid cannot be opened when the apparatus is switched on and such that the apparatus cannot be switched on when the sliding lid is open, thus ensuring that the rotary switch cannot be operated while the apparatus is in use. One end of the cage houses canister guide 17, a valve block 18, an ozone generator 19, a circuit board 20 and a rotary switch 21.

The canister guide 17 can hold separate pressurized cylinders containing oxygen, an olefin such as butene, and water vapor. The olefin feeds directly to valve 18a. The water vapor cylinder (if present) feeds directly to valve 18b. The oxygen feeds directly to valve 18c and thence to the ozone generator.

FIGS. 2d to 2f illustrate an ozone generator which comprises an electrically conductive ground plate 25, a plate 26 formed from an insulating material, such as calcium silicate, in which a spiral groove 28 is machined, and a cover plate 27 formed from an insulating material, such as calcium silicate. A conductive element, such as a copper wire 29 is laid in the spiral groove 28. The copper wire is connected to a high voltage capacitative discharge unit having a typical output of up to 15 kV at 1 kHz. The unit is preferably operated from a 12 volt battery supply but may be operated at any convenient voltage.

The insulating cover plate 27 includes an inlet 30 and an outlet 31. The inlet 30 is connected to a source of oxygen, which in this example is the oxygen canister. Oxygen is delivered to the centre of the spiral groove 28 along which it travels. The charged copper wire 29 ionizes the oxygen to generate ozone (0₃). The generated ozone exits through the outlet 31 located towards an outer extremity of the spiral. The three plates 25, 26 and 27 are clamped together by bolts 32 at each corner of the plates. For the sake of clarity, only one bolt 32 is shown.

FIGS. 2g to 2i illustrate an alternative form of ozone generator 33, which comprises an electrically conductive core 34, a first insulating tube 35 on the outer surface of which a groove 37 is machined, and a second insulating tube 36 which surrounds the tube 35. The first and second insulating tubes may be formed from calcium silicate. An electrically conductive wire 38 sits in the groove 37, and is connected to a capacitative discharge unit as described above. The electrically conductive core 34, which in the example is ferrous, is connected to ground. Oxygen enters the one end of the spiral 37 and is ionized as it moves along the spiral exiting from the other end of the spiral as ozone (0₃). The advantage of using the construction illustrated in FIGS. 2g to 2i is that the same length of spiral and hence copper wire can be located in a smaller volume than can be achieved with the construction of FIGS. 2d to 2f.

Referring now to FIGS. 3a and 3b, there is shown a hood 6. As shown in FIG. 1, the hood is connectable to a tube that delivers ozone and olefin. The function of the hood is to mix ozone and gaseous olefin to generate hydroxyl radicals and to dispense the generated radicals onto a surface. The hood may include contact sensors which detect that the sides of the hood are in contact with a surface, so that discharge of gases can only occur when the hood is in contact with a surface. A hand operated trigger provides for the hood to disinfect surfaces with high concentrations of hydroxyl radicals, i.e. the rate of delivery of ozone and olefin to the hood is increased.

FIGS. 4a to 4g, illustrate components of a shroud into which a person's hands may be inserted. Once inserted the air-moving device, if fitted, can remove most of the air from the shroud. The ozone and olefin is mixed in the chamber and then fed into the shroud for a prescribed period to ensure that the preponderance of pathogens on the user's hands are killed.

FIG. 4a is a cross-section of a plate 50 which is shaped to receive the shroud illustrated in FIG. 4c to 4e. The plate 50, which in the example is cast from aluminum provides brackets 52 and 53 which engage with the handle 7 of the chassis 1 (see FIG. 1). The plate 50 includes a planar surface 51 upon which a shroud of the type shown in FIGS. 4c to 4e sits. The planar surface 51 includes projections 56 so shaped and located as to engage with depressions in the shroud. The plate 50 includes a bracket comprising spaced apart plates 54. Each plate 54 includes an aperture 55 through which a pin may be passed. A clamping member as illustrated in FIGS. 4f and 4g sits between the plates 54 and is pivotable about the pin extending between the two plates.

FIGS. 4c to 4d illustrate a hand shroud template 60, which is vacuum formed from a plastics material. The shroud template 60 comprises top and bottom halves of the hand shroud, which are put together by folding along the line X and electro-staking edges 61 and upstand elements 62.

The upstand elements 62 on one side of the line X are electro-staked to corresponding upstand elements 62 on the other side of the line X. The resulting upstands form finger separators. It is important that the fingers are separated during the disinfection process so that the hydroxyl radicals are not prevented from entering any areas of the hand which might harbor pathogens.

The shroud template 60 further includes a plurality of raised dimples 63. The purpose of these raised dimples is to ensure that air and hence hydroxyl radicals can move freely under the palm of a hand located in the shroud.

The shroud template 60 also includes opposing halves 64 of inlet chambers through which hydroxyl radicals are fed into the shroud. When the template 60 is folded about line X, the two halves 64 of the inlet chambers meet, thereby forming a chamber. Extending from each chamber are conduits 65, similarly formed in halves. The conduits 65 open up into the inside of the glove elements of the shroud providing for hydroxyl radicals to disinfect hands in the glove elements.

The shroud template 60 also includes opposing halves 66 of an outlet chamber, which may be is connected to an air moving fan to remove air containing destroyed pathogens from the gloves of the shroud. The outlet chamber is connected to the gloves by conduits 67.

FIGS. 4f and 4g illustrate the clamping member 70. The clamping member 70 includes a handle 70, a pair of spaced apart plates 72 each including an aperture 73 through which a pin is passed to attach the clamping member to the plate 51, and a bracket 74 so shaped and dimensioned as to engage with a handle of the chassis 1. The clamping member 70 also includes piercing means 75 and 76, which are arranged to pierce the chambers 66 and 64 respectively.

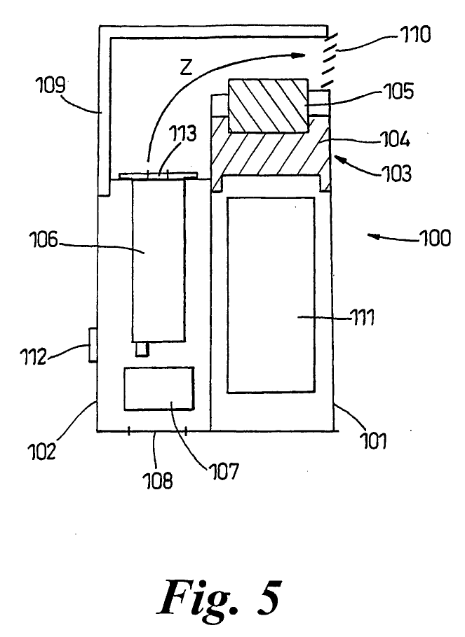

In FIG. 5 there is illustrated a hand portable device 100 for generating hydroxyl radicals. The device 100 comprises two compartments 101 and 102. Compartment 101 houses a battery power pack 111, the battery preferably being re-chargeable, (which provides power for an ozone generator and a fan), and mounting an olefin source 103. The olefin source includes a bath of liquid olefin 104 and a wick 105 saturated with the liquid olefin. The compartment 102 houses an ozone generator 106 of the type illustrated in FIGS. 2d to 2f. A fan 107 draws air through a grille 108, directing the said air into the base of the ozone generator 106 where it is ionized to ozone. A stream of ozone Z exits the ozone generator 106 through an outlet 113. A cap 109 clips onto the top of the first and second compartments 101, 102. The cap directs the stream of ozone Z over the olefin saturated wick to generate hydroxyl radicals, which radicals pass through a grille 110 to atmosphere. The ozone generator 106 is a consumable component and can be removed from the compartment 102. Similarly, the olefin source 103 is a consumable component and can be removed from the compartment 101 and replaced. The device illustrated in FIG. 5 may be mounted in a pig furrowing pen, by a hospital bed, or on the clothes of a person.

An alternative apparatus is a personal device which uses atmospheric air as its supply of oxygen. The device is sized to fit in a jacket pocket and comprises an ozone generator, which may be of the type illustrated in FIGS. 2d to 2f, a power source in the form of a battery pack, a source of olefin (such as a small pressurized canister), a fan to force air towards and through the ozone generator, and a mixing chamber where the generated ozone and olefin come together and are released to the atmosphere. The mixing chamber may include an outlet in the form of a jet to direct the hydroxyl radicals in a desired direction.

The invention provides a means of generating hydroxyl radicals in a controlled manner, allowing them to be used for the purposes of disinfecting enclosed spaces, cleaning surfaces including flat surfaces and parts of the body, and prophylactically, i.e. releasing hydroxyls intermittently into an enclosed space. The invention also provides a personal device suitable for use in an enclosed environment. The personal device may be used to create a micro-environment around the person in which the concentration of hydroxyl radicals is raised in comparison to the concentration in the remainder to the enclosed environment. The apparatus may be used in hospital wards, waiting rooms, surgeries, veterinary surgeries, operating theatres, aircraft, trains, hotels, ships, animal barns, and for personal security.

The effects of releasing hydroxyl radicals into the atmosphere include: deodorization, killing bacteria, viruses, spores and fungi.

In accordance with the provisions of the patent statutes, the principle and mode of operation of this invention have been explained and illustrated in its preferred embodiments. However, it must be understood that this invention may be practiced otherwise than as specifically explained and illustrated without departing from its spirit or scope.

What is claimed is:

1. Apparatus for generating hydroxyl radicals comprising:
a source of oxygen;
a source of olefin;
an ozone generator;
a control unit; and
mixing means,
wherein the ozone generator is connectable to the source of oxygen, and wherein generated ozone and olefin are delivered to the mixing means to generate hydroxyl radicals.

2. Apparatus according to claim 1, wherein the said apparatus is portable.

3. Apparatus according to claim 1, wherein the source of oxygen is substantially pure oxygen.

4. Apparatus according to claim 3, wherein the substantially pure oxygen is contained in a pressurized canister.

5. Apparatus according to claim 1, wherein the source of oxygen is atmospheric air.

6. Apparatus according to claim 1, wherein the olefin is contained in a pressurized canister.

7. Apparatus according to claim 1, further including a source of humidity, and wherein humidity is delivered to the mixing means.

8. Apparatus according to claim 7, wherein the source of humidity is water vapor.

9. Apparatus according to claim 8, wherein the water vapor is stored in a pressurized canister.

10. Apparatus according to claim 7, wherein the source of humidity includes a ferrous salt.

11. Apparatus according to claim 1, further comprising a hood having an open face for the dispensing of hydroxyl radicals, wherein ozone and olefin are delivered to the hood, wherein the hood constitutes the said mixing means.

12. Apparatus according to claim 11, wherein the hood includes sensors, the senses indicating whether edges of the hood are in contact with a surface, the sensors sending a signal to the control unit and wherein the control unit prevents the delivery of ozone and/or olefin to the hood if the hood is not in contact with a surface.

13. Apparatus according to claim 1, further including a manually operated trigger mechanism, said mechanism being connected to the control unit such that when the trigger is activated the control unit increases the rate of production of ozone and/or delivery of olefin.

14. Apparatus according to claim 1, further comprising a hand disinfection unit, the unit including a shroud into which hands may be inserted and at least one mixing chamber.

15. Apparatus according to claim 14, wherein the shroud includes two spaced-apart chambers, each shaped and dimensioned to receive a hand.

16. Apparatus according to claim 5, wherein the apparatus is a hand-held device.

17. Apparatus according to claim 16, wherein the device includes a fan to deliver air to the ozone generator and generated ozone to the mixing means.

18. An ozone generator comprising a conductive layer, a first insulating layer and a second insulating layer, and a spiraled electrically conductive element located between the first and second insulating layers.

19. An ozone generator according to claim 18 wherein the spiraled electrically conductive element sits in a spiraled grove in a surface of one of the first and second layers.

20. An ozone generator according to claim 19 wherein the spiraled grove is machined into the surface.

21. An ozone generator according to claim 18, wherein the conductive element is connected to a source of power and the conductive layer is connected to ground.

22. An ozone generator according to claim 21, wherein the source of power is a high voltage capacitative discharge unit.

23. An ozone generator according to claim 18, wherein the conductive layer, the first insulating layer and the second insulating layer are each a planar element, the first insulating layer being sandwiched between the conductive layer and the second insulating layer.

24. An ozone generator according to claim 18, wherein the conductive layer is a rod having substantially circular in cross-section, the first insulating layer tubular, and slides over the rod, and the second insulating layer is a tubular and slides over first insulating layer.
